(19) United States
(12) Patent Application Publication
(10) Pub. No.: US 2024/0216595 A1
(43) Pub. Date: Jul. 4, 2024

(54) PORTABLE HEMOFILTRATION COMPONENTS, SYSTEMS, AND METHODS

(71) Applicant: University of Washington, Seattle, WA (US)

(72) Inventors: Shen Ren, Seattle, WA (US); Dayong Gao, Seattle, WA (US); Ye Jin, Seattle, WA (US)

(73) Assignee: University of Washington, Seattle, WA (US)

(21) Appl. No.: 18/558,591

(22) PCT Filed: May 3, 2022
(86) PCT No.: PCT/US2022/027489

§ 371 Date: Nov. 2, 2023

Related U.S. Application Data
(60) Provisional application No. 63/183,482, filed on May 3, 2021.

(51) Int. Cl. A61M 1/16; A61M 1/34; A61M 1/36

(52) U.S. Cl. CPC A61M 1/1696; A61M 1/3401; A61M 1/3649; A61M 2205/3331; A61M 2205/3368

(57) ABSTRACT

A portable hemopurification system can remove one or more uremic toxins from blood of a patient. The hemopurification system includes a dialyzer with a dialyzing membrane. Blood from a patient is provided to the dialyzer on one side of the dialyzing membrane, and a negative-pressure pump applies negative pressure on the opposite side of the dialyzing membrane to provide ultrafiltration of uremic toxins from the blood. The system includes a replacement fluid line to deliver replacement fluid to the blood. The system includes load cells to measure the replacement fluid flow, effluent flow, and returned blood flow. The systems uses the fluid weights for the control of pumps to provide stable flow through the system.

### CROSS-REFERENCE(S) TO RELATED APPLICATION(S)

This application claims the benefit of U.S. Provisional Application No. 63/183,482, filed on May 3, 2021, the entire contents of which are fully incorporated herein expressly by reference.

### BACKGROUND

Kidney disease is one of the most severe chronic diseases that could lead to multi-organ failure and is the ninth leading cause of the death in United States. Every one in seven American adults develops chronic kidney disease and remains a devastating medical, social, and economic problem for patients, families, and communities worldwide. The only treatment available for patients who progress to end-stage renal disease (ESRD) are hemodialysis. Millions who need these treatments do not receive them. Only two million patients are on dialysis worldwide leaving millions more to die every year from lack of access to this life-saving therapy.

Current dialysis methods rely on the diffusion-based technology that requires large amount of purified water (approximately 120 liters/session) to remove renal toxins at relatively low efficiency. ESRD patients generally spend 4-6 hours/session, 3-5 sessions/week on dialysis treatment at clinics due to the large use of the dialysate. Despite the inconvenience and time consuming for patients on the commute to the clinics, the poor performance of diffusive method leads to the 5-year survival rate under 50%. The average life expectancy is only 3 years, and risks of complications from infections, blood clots, and vascular access failure remain exceedingly high. Moreover, the complicated system and the large water usage make dialysis treatment's cost extraordinarily high that exceed $42 billion annually. In other words, with only 1% of Medicare population, dialysis care spends more than 7% of the total budget.

Liver disease is the 11^{th} leading cause of death accounting for 2 million deaths each year globally. Deaths are either caused by acute liver failure, commonly as a result of drug toxicity, or acute on chronic liver failure (AOLF) usually with the involvement of other organ systems. About one third of patients hospitalized with cirrhosis develop acute worsening of liver function with poor prognosis. Prognosis with three or more organ system failure is extremely poor with 28-day mortality of 75% to 90%. The outcome is particularly poor in those who have renal failure requiring dialysis, with survival probability of <10% even with state of the art medical support. The current pandemic of SARS-COV-2 has increased the risk of liver damage, thus increasing the risk of serious morbidity and mortality.

Liver transplantation is the only long term option to prevent deaths, however, only 10% of the global need for transplantation is currently met. Most patients with liver failure die while waiting for an organ. Thus, there is a need for new methods of therapeutic support to those who are waiting for a transplant, bridge-to-transplantation and those in whom recovery of liver function is possible where such a device can be used as bridge-to-recovery.

As early as 1960s various extracorporeal techniques were used to improve prognosis in liver failure patients with renal and other system involvement. These therapeutic interventions included kidney dialysis, cross circulation with kidney failure patients with normal liver and charcoal hemoperfusion. All were unsuccessful as bridge-to-transplant or bridge-to-recovery. Liver failure leads to the accumulation of toxins that are bound to albumin thus are difficult to dialyze. Theses toxins are considered to be responsible for many complications associated with advanced liver failure such as encephalopathy, hepato-renal syndrome, immune system abnormalities and cardiovascular abnormalities. From the 1990s several systems based on the concept of using albumin containing dialysate solution to remove albumin bound toxins, as a consequence of liver failure, were developed. The albumin systems that have been used include the Single-Pass Albumin Dialysis system (SPAD), and the Molecular Adsorbent Recirculating System™ (MARS™). The other extracorporeal device, the Fractionated Plasma Separation and Adsorption system—FPSA (Prometheus™) does not use albumin but uses plasma filtration and regeneration methods. Currently, MARS is the most commonly used method in the United States.

The MARS has many limitations including its high cost, limited ability to achieve simultaneous kidney dialysis and extracellular volume control by ultrafiltration. Randomized studies reviewing the results of the MARS use have shown some mortality benefit however there are also reports of hemodynamic instability with its use. In AOCLF, the use of MARS has not increased the probability of receiving transplantation compared to the use of standard medical treatment without MARS.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one embodiment, a portable and automated hemopurification system configured to remove one or more uremic toxins from blood of a patient, comprises the following elements:
a primary dialysis circuit comprising
a blood inlet line configured to deliver blood from the patient;
a blood outlet line configured to return blood to the patient; and
a dialyzer with a dialyzing membrane, an inlet end that receives blood provided by the blood inlet line, and an outlet end that provides blood towards the blood outlet line;
a negative-pressure pump configured to apply negative pressure across the dialyzing membrane, to provide ultrafiltration of uremic toxins from the blood;
a replacement fluid line configured to deliver replacement fluid to the blood; and
pumps configured to drive blood and fluidic flow in the system.

In one embodiment, the negative-pressure pump applies a negative pressure across the dialyzer membrane to draw the uremic toxins across the dialyzing membrane.

In one embodiment, the dialyzer includes a first compartment and a second compartment separated by the dialyzing membrane, and the first compartment includes the inlet for the blood inlet line and the outlet for the blood outlet line, and the second compartment includes no inlet and an outlet for a line to the negative-pressure pump,

In one embodiment, the system operates using Continuous Veno-Venous Hemofiltration (CVVH), intermittent veno-venous hemofiltration, continues arteriovenous hemofiltration.

In one embodiment, dimensions of a suitcase containing the portable hemopurification system are equal to or less than 55 cm×40 cm×20 cm.

In one embodiment, a weight of the portable hemopurification system is 25 pounds or less.

In one embodiment, the portable hemopurification system further comprises a real-time flowrate and weight management system, comprising:
a replacement fluid load cell configured to measure weight of replacement fluid at the start, end, and throughout the entire treatment;
an effluent load cell configured to measure weight of effluent produced by the negative-pressure pump at the start, end, and throughout the entire treatment;
a total weight load cell configured to measure the total weight of replacement fluid and effluent at the start, end, and throughout the entire treatment; and
a controller configured to use changes in weight from the replacement fluid load cell, the effluent load cell, and the total weight load cell to determine:
a replacement fluid rate;
an effluent rate;
a net-filtration rate; and
combinations thereof.

In one embodiment, a control system tracks the replacement fluid rates, effluent rates, and checks any variations from the total weight and triggers an alarm if there are any variations.

In one embodiment, the control system permits corrective measures to be taken automatically.

In one embodiment, the portable hemopurification system further comprises a disposable cassette system that contains the dialyzer, pressure sensor connectors, temperature sensor connectors, anti-coagulant connectors, and at least portions of the blood inlet line and the blood outlet line.

In one embodiment, the portable hemopurification system further comprises a non-disposable layer that includes sensors and electrical components configured to monitor and operate the system.

In one embodiment, the portable hemopurification system further comprises sensors selected from pump sensors, blood leak detection (BLD), air-bubble detection (ABD), pressure sensors, load cells, temperature sensors, pinch valves, and combinations thereof.

In one embodiment, the portable hemopurification system further comprises an automatic priming, rinse-back system.

In one embodiment, the replacement-fluid line further comprises a fluid warmer.

In one embodiment, the medical-fluid warmer comprises a disposable heating cassette comprising a serpentine fluidic path in thermal communication with at least one heating element.

In one embodiment, a fluid warmer comprises a disposable heating cassette comprising a serpentine fluidic path in thermal communication with at least one heating element.

In one embodiment, the fluid warmer further comprises an insulation top cover, an insulation bottom cover, a top heating pad juxtaposed beneath the top insulation top cover, a bottom heating pad juxtaposed above the insulation bottom cover, a top copper conductive panel juxtaposed below the top heating pad, a bottom copper conduction panel juxtaposed above the bottom heating pad, wherein surfaces of the top and bottom copper conductive panels facing one another include the serpentine flow channel.

In one embodiment, the fluid warmer is configured for uses as Replacement Fluid Warming During Continuous Renal Replacement Therapy (CRRT); Emergency Department (ED) IV Fluid Warming; or a blood warmer.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic illustration of one embodiment of a hemopurification system;

FIG. 2 is a diagrammatical illustration of one embodiment of a portable hemopurification system of FIG. 1;

FIG. 3 is a diagrammatical illustration of one embodiment of the portable hemopurification system of FIG. 2 showing the component layers;

FIG. 4 is a diagrammatical illustration of one embodiment of the flowrate and weight management layer;

FIG. 5 is a schematic illustration of one embodiment of the control system for the hemopurification system;

FIG. 6 is a graph showing the effluent and replacement fluid weights;

FIG. 7 is a diagrammatical illustration of one embodiment of the disposable cassette layer;

FIG. 8 is a diagrammatical illustration of one embodiment of the sensors and electrical components layer;

FIG. 9A is a diagrammatical illustration of a component for connecting the disposable cassette layer and the sensors and electrical components layer;

FIG. 9B is a diagrammatical illustration of a component for connecting the disposable cassette layer and the sensors and electrical components layer;

FIG. 10 is a schematic illustration of one embodiment of a hemopurification system with a liver module; and

FIG. 11 is a schematic illustration of one embodiment of a hemopurification system with a liver module and a lung module;

FIG. 12 is a schematic illustration of the hemopurification system of FIG. 1 in the priming step;

FIG. 13 is a schematic illustration of the hemopurification system of FIG. 1 in the rinse step;

FIG. 14 is an example of the stability of the blood flow using the automatic control system; and

FIG. 15A is a schematic illustration of one embodiment of a medical-fluid warmer; and

FIG. 15B is a schematic illustration of one embodiment of a medical-fluid warmer.

### DETAILED DESCRIPTION

In one embodiment, the disclosure relates to a portable hemopurification system that provides efficient, flexible, easy access, less water usage, user-friendly, and affordable blood purification treatment. This system will enable higher uremic toxin removal rate, improve the long-term longevity and quality of life for end stage renal disease (ESRD) patients.

In one embodiment, the hemopurification system is based on convection only toxin removal. Convection only toxin removal can lead to advantages that have not been possible previously using a single device. Such advantages may include simplicity of the system, decreasing the size and weight, and significantly reducing the usage of purified water. The performance characteristics of the disclosed hemopurification system may enable transformation of the traditional hemodialysis treatment from clinic-based to a flexible therapy that is possible most anywhere, at any time, making it a strong medical device at multiple application fields.

By carefully designing the testing methodology and protocol to maintain the patient's average concentration of the uremic toxins, this device can effectively improve patient's comfort level and long-term longevity. Additionally, in one embodiment, a medical-fluid warmer is integrated into the system to achieve temperature control during the treatment. Overall, the hemopurification system has potential application as a 1. portable/home-based blood purification device: to remove the uremic toxins for ESRD patients based on daily, convection only technology; 2. mobile continuous renal replacement therapy (CRRT) machine in Intensive Care Unit (ICU) and Emergency Room to achieve long term, continuous renal therapy with real-time monitoring and automatic controlling system; and 3. a clinical fluid warming tool to quickly and accurately heat up the medical fluid to ideal temperature with a low power, compact system.

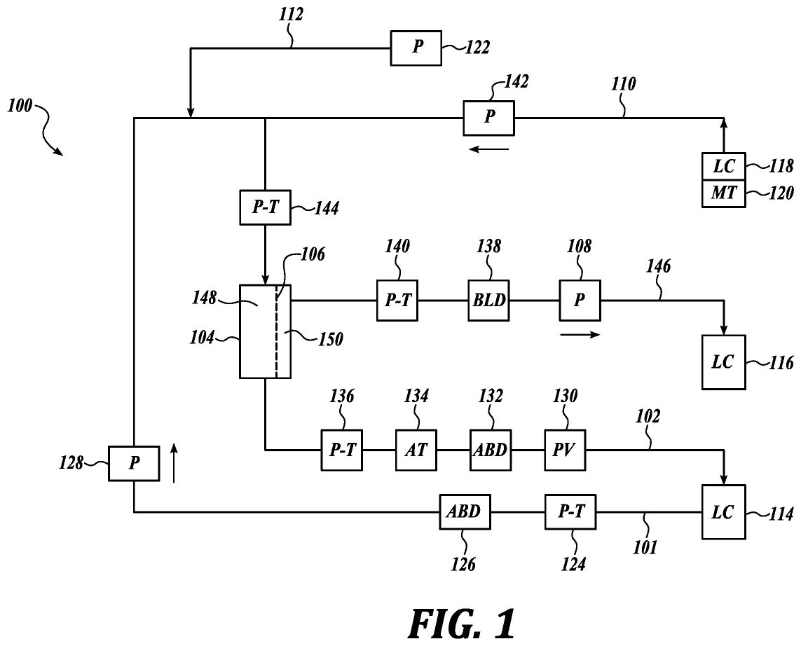

FIG. 1 is a schematic illustration of one embodiment of the hemopurification system 100 of this disclosure. The portable hemopurification system 100 is configured to remove one or more uremic toxins from blood of a patient.

The system 100 includes convection-based ultrafiltration to achieve the high uremic toxin removal rate. FIG. 1 includes a portable and automated hemopurification system configured to remove one or more toxins from blood of a patient and treat imbalanced body fluid in blood caused by disease of vital organs, such as liver, kidney, and lung.

In one embodiment, the hemopurification system 100 includes a primary dialysis circuit comprising a blood inlet line 101 configured to deliver blood from a patient to the dialyzer 104. The system 100 includes a blood outlet line 102 configured to return blood to the patient from the dialyzer 104. The system 100 includes the dialyzer 104 with a dialyzing membrane 106. The dialyzer 104 includes an inlet end that receives the blood provided by the blood inlet line 101 and an outlet end that provides blood towards the blood outlet line 102. The system 100 includes a negative-pressure pump 108 configured to apply negative pressure across the dialyzing membrane 106 to provide ultrafiltration of uremic toxins from the blood. The system 100 includes a replacement fluid line 110 configured to deliver replacement fluid to the blood prior to or after the dialyzer 104. In another embodiment, the replacement fluid can be configured to deliver replacement fluid after the dialyzer. The system can, therefore, operate in pre-dialyzer mode and/or post dialyzer mode. The system includes one or more pumps 128 configured to drive blood through the system. The system includes that replacement fluid pump 142 and the pump 122 to inject anticoagulant is necessary.

The blood inlet line 101 usually comes from a from a vein in the patient, and the blood outlet line 102 is usually returned to an artery in the patient. The replacement fluid line 110 is connected to the blood inlet line 101 before the blood enters the dialyzer 104. The replacement fluid can be stored within a container, such as a bag. In one embodiment, the replacement fluid container rests atop the load cell 118. The replacement fluid can be a bicarbonate buffered solution or a lactate-buffered solution or any conventional replacement fluid used in continuous veno-venous hemofiltration (CVVH).

The dialyzer 104 can include a housing to hold the dialyzing membrane 106. The dialyzing membrane 106 separates two compartments 148, 150 of the housing. The dialyzing membrane 106 can be a semi-permeable hollow fiber membrane or a semi-permeable flat membrane, or any combination. The dialyzing membrane 106 can include any conventional dialyzing membrane known in the art. A pore size of the dialyzing membrane 106 can be selected to remove large molecules. The dialyzing membrane 106 separates the compartment 148 that receives the blood from the blood inlet line 101 from the compartment 150 to which the negative-pressure pump 108 is connected. The dialyzing membrane 106 allows toxins in the blood and fluids to be transferred through the dialyzing membrane 106 from the compartment 148 into the compartment 150. In one embodiment, the compartment 148 does not include an inlet line and has a effluent outlet line 146 to which the pump 108 is connected.

The pump 108 is configured to create negative-pressure or a pressure differential across the dialyzing membrane 106 to force the toxins and fluid across the membrane 106 for the ultrafiltration of uremic toxins. The higher the ultrafiltration rate, the higher the toxin removal rate. The system operates using Continuous Veno-Venous Hemofiltration (CVVH). With the negative-pressure pump 108, the hemopurification system 100 does not rely on diffusion for the transport of toxins across the dialyzing membrane 106, which simplifies the dialyzer 104 design to have only one dialysate port (dialysate inlet is not in use). The patient receives blood returned from the dialyzer 104 through the blood outlet line 102.

In one embodiment, the returned and filtered blood is optionally warmed by a medical-fluid warmer 120 on the replacement fluid line 110. The medical-fluid warmer 120 can warm the replacement fluid before being combined with the blood. The returned blood from line 102 is weighed by a load cell 114, prior to reintroduction into the patient. The blood loop begins with blood being removed from the patient through the blood inlet line 101 for introducing into the dialyzer 104.

In one embodiment, the hemopurification system 100 is configured to add anticoagulants. The anticoagulant line 112 is connected to the blood inlet line 101. A pump 122, such as a syringe pump, is used for the injection of the anticoagulant, when necessary, to avoid blood clotting during the treatment. The injection rate of the syringe pump 122 can be monitored and controlled by an embedded automatic control system to control the flowrate of anticoagulants.

The replacement fluid is directly injected through replacement fluid line 110 to the blood inlet line 110 before the dialyzer 104. Since the efficient toxin removal achieved by ultrafiltration, the usage of replacement fluid is significantly reduced from about 120 liters in dialysis to about 20 liters in the present hemopurification system. With the reduced volume requirements, the fluid would be delivered to the patients without additional on-site water purification system, which is a cost-saving advantage of the portable hemopurification treatment and system. The replacement fluid line 110 includes a load cell 118 to measure the weight of replacement fluid being delivered.

The hemopurification system 100 may also include monitoring instrumentation. For example, the blood inlet line 101 can include an instrument cluster 124 that can measure temperature and pressure. The blood inlet line 101 can also include an air bubble detector 126.

The blood outlet line 102 may also include an instrument cluster 136 including a temperature and pressure sensor. Additionally, the blood outlet line 102 may include an air trap 134 an air bubble detector 132 and an automatic valve 130, such as a pinch valve.

In one embodiment, the effluent line 146 may include an instrument cluster 140 including a pressure and temperature sensor. In one embodiment, the effluent line 146 may include a blood leak detector 138. The effluent that is removed through the effluent line 146 is stored in an effluent container, such as a plastic bag. In one embodiment, the effluent container can rest atop the load cell 116 to weigh the amount of effluent being removed.

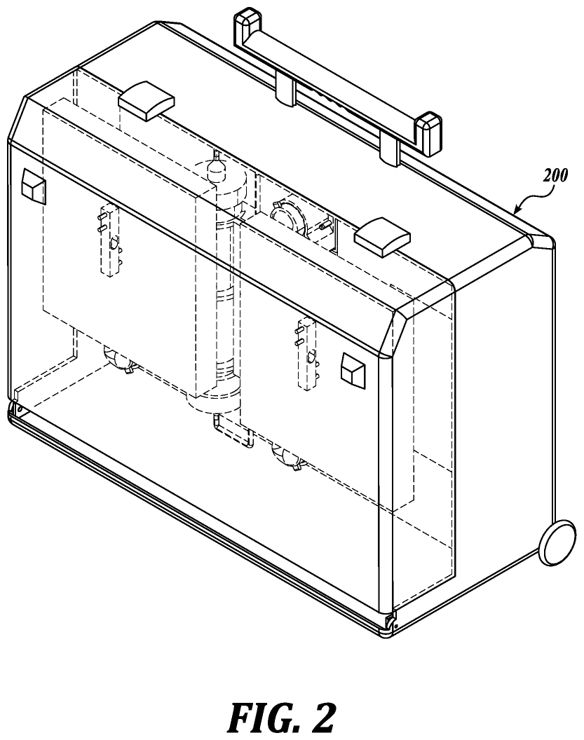

FIG. 2 is an illustration showing one embodiment of the hemopurification system 100 designed to be portable. In one embodiment, the hemopurification system 100 is configured to fit within a generally rectangular case 200. The case 200 can include different layers that comprise the different components of the system of FIG. 1 that can be packed together. In one embodiment, the different layers can be disposable or non-disposable layers. In one embodiment, the exterior dimensions of the case 200 in the packed mode can be limited to 55 cm by 40 cm by 20 cm (21.6 inch by 15.7 inch by 7.9 inch), for example. The size is suitable to fit in the carry-on compartment on a plane. In one embodiment, the hemopurification system in case form weighs less than 25 pounds that can be easily carried with one hand. The compact system allows patients to bring and setup the device at anywhere, at any time (home, work, school, travel, etc.) to achieve flexible treatment.

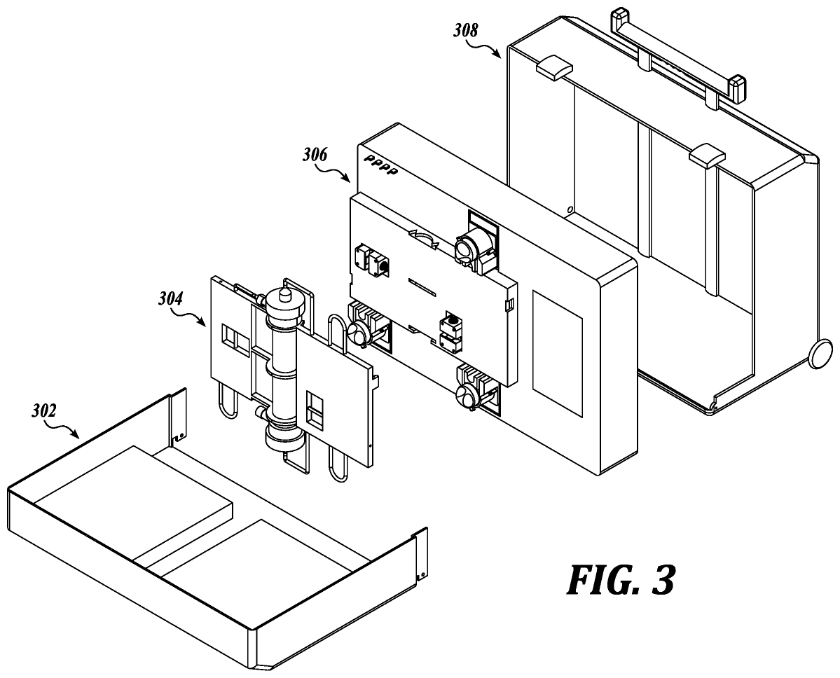

FIG. 3 is an illustration of the different component layers of the case 200 comprising the hemopurification system 100. The component layers include a real-time flowrate and weight management layer 302, a disposable cassette layer 304, sensors and electrical components layer 306, and a mobility case layer 308. As described further herein, the disposable cassette layer 308 can plugged into the sensors and electrical components layer 306.

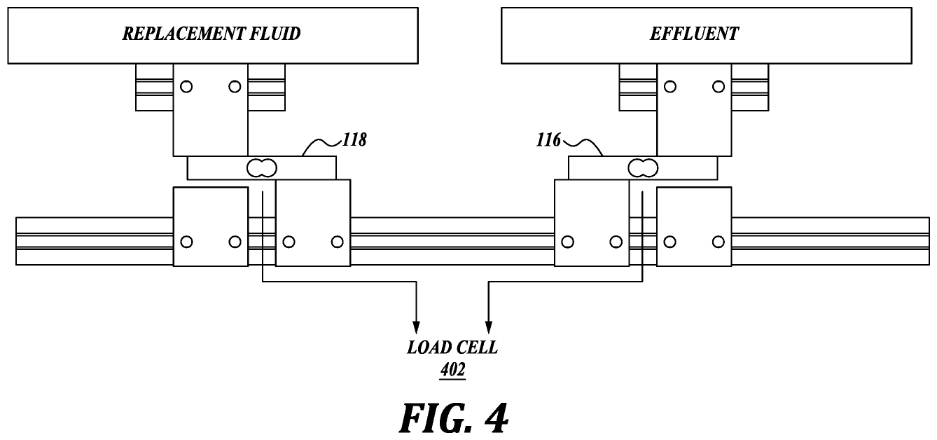

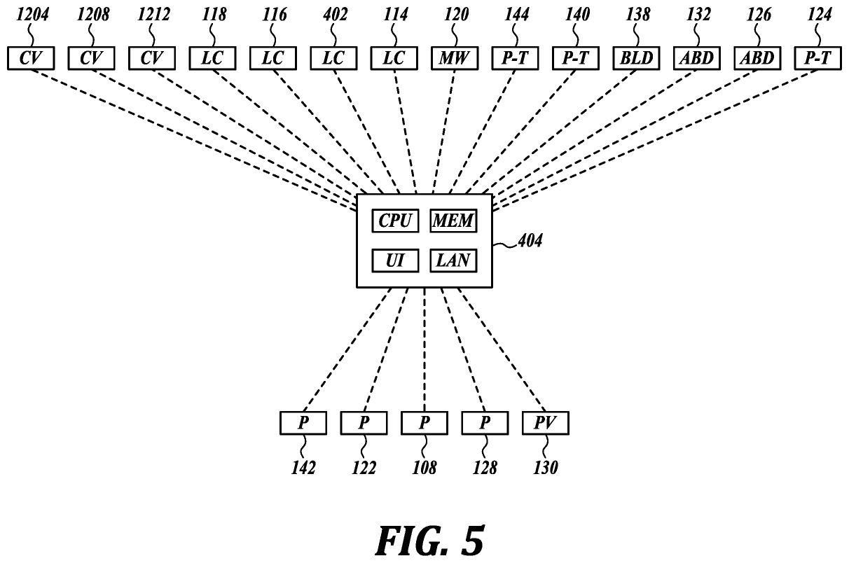

FIG. 4 is schematic illustration of the flowrate and weight management layer 302. In one embodiment, the flowrate and weight management layer 302 uses three load cells. Load cell 116 is used for measuring the weight of the effluent from the effluent line 142. For example, when a bag is used for the effluent container, the bag will rest atop the load cell 116. Load cell 118 is used for measuring the weight of the replacement fluid in the replacement fluid line 110. For example, when a bag is used for the replacement fluid container, the bag will rest atop the load cell 118. A third load cell 402 is used for measuring the combined weight of the replacement fluid and the effluent. The three load cells 116, 118, and 402 are designed to allow the user to put weight on top that benefit (i) avoid weight lifting to a high elevation, (ii) balance the entire system during the treatment, (iii) precise and fast response from the load cell to achieve real-time measurements. The flowrate and weight management layer 302 may also include an automatic control system 404 illustrated and described herein in association with FIG. 5. In one embodiment, the control system 404 is used to integrate the weight over time from the load cells. Therefore, the control system 404 can provide the weights over time as the system operates. For example, the control system 404 measure and record weight of replacement fluid at the start, end, and throughout the entire treatment; measure and record weight of effluent produced by the negative-pressure pump at the start, end, and throughout the entire treatment; and measure and record the total weight of replacement fluid and effluent at the start, end, and throughout the entire treatment.

FIG. 5 is a schematic illustration of the sensors, pumps, valves, and the like, that communicate with and/or are controlled by the control system 404.

The control system 404 can be implemented using a central processing unit, a memory, a user interface, such as keyboard, mouse, touch display, and the like. The control system 404 may be connected to a local area network via a hard-wired or wireless connection. In one embodiment, the control system 404 includes at least one processor and a system memory connected by a communication bus. Depending on the exact configuration and type of device, the system memory may be volatile or nonvolatile memory, such as read only memory (“ROM”), random access memory (“RAM”), EEPROM, flash memory, or similar memory technology. Those of ordinary skill in the art and others will recognize that system memory 904 typically stores data and/or program modules that are immediately accessible to and/or currently being operated on by the processor. In this regard, the processor may serve as a computational center of the control system 404 by supporting the execution of instructions.

The control system 404 receives information from the sensors of the hemopurification system, and implements a control program to control the flowrates in the system to maintain effectiveness. Other control programs may be used to monitor the safety of the hemopurification system. The control programs are a series of instructions that can be hardwired or written in a programming language. The control programs can be stored in any type of computer readable medium or computer storage device and be stored on and executed by one or more general purpose computer processors. The computer readable medium can be a medium such as flash memory, random access memory (RAM), hard disk drives, and/or the like.

In one embodiment, the safety and effectiveness of the hemopurification system is monitored in real-time through the integrated sensors, including but not limited to the load cells 118, 116, 402, 114, the pressure-temperature sensors 144, 140, 124, the blood leak detector 138, the air bubble detectors 132, 126. In one embodiment, the control system 404 is configured to calculate the various flowrates of the blood inlet line, the replacement fluid line, and the effluent line, and make adjustments to the flows via pumps 128, 108, 142. In an embodiment, the control system 404 of the portable hemopurification system can keep track the replacement fluid rates, effluent rates, and checks any variations from the total weight. An alarm will be triggered if there is any variations. Such variations may include a problem with pumps and/or a leak in the system, for example. In an embodiment, the portable hemopurification system can take corrective action when the control system 404 determines a variation of the weights, for example, the control system 404 can close the valve 130. However, other automated valves may be included throughout the system.

The control system 404 can be further configured to check the testing parameters throughout the treatment. The automatic control system 404, may (i) check pump's RPM by an encoder to ensure the flow rate within the preset range, (ii) when an air bubble is collected by the air trap 134, any remaining air bubble detected by the air bubble detector 132 can trigger the release of the pinch valve 130 to cutoff the entire flow in the blood line 102, (iii) monitor and record pressure at multiple locations (prior and after the dialyzer 104, ultrafiltration-side, prior and after the patient) by the pressure sensors to monitor any potential clotting or leaking, and observe the performance of the ultrafiltration through the pressure change of the transmembrane pressure.

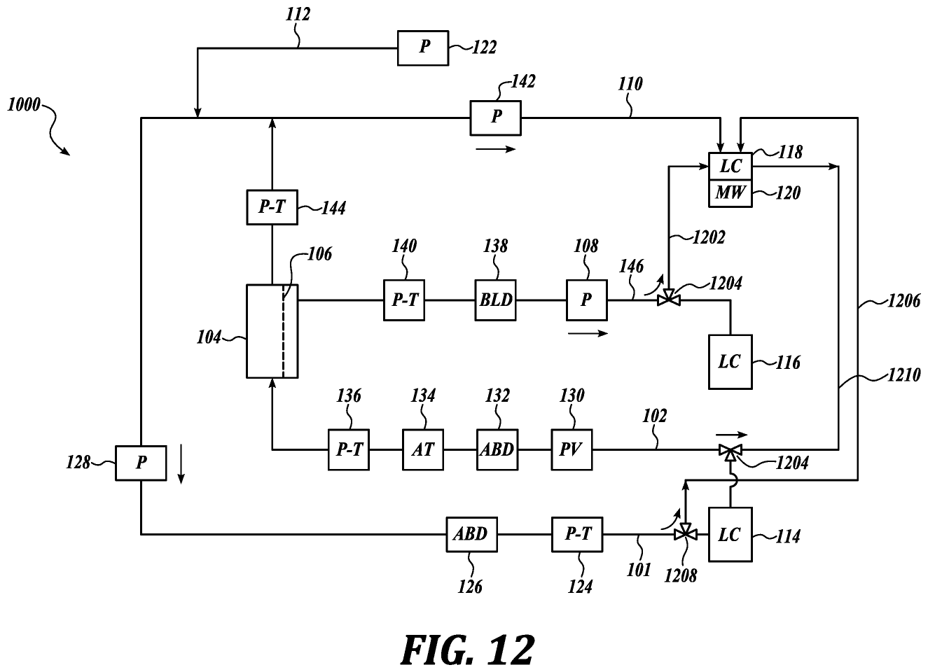

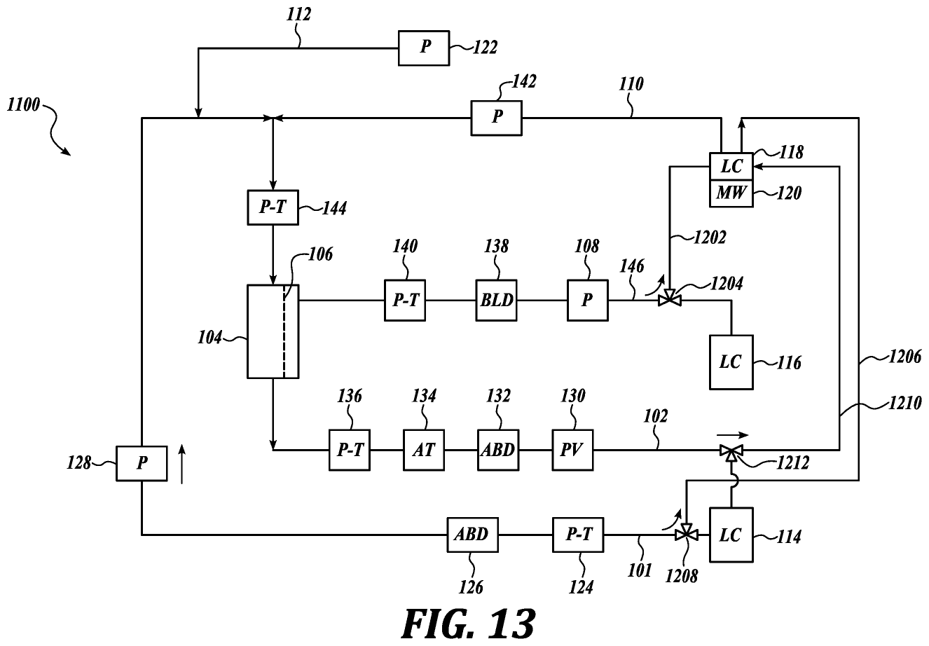

As described herein, the control system 404 can be further configured to perform an automatic priming and a rinse-back process. The automatic priming and rinse-back process is described and illustrated in FIGS. 12 and 13. During the priming and a rinse-back process, the control system 404 is configured to control the flowrate and the direction of flow using pumps 128, 142, 108, and a series of valves, such as three-way control valves 1204, 1208, and 1212.

In one embodiment, real-time monitoring of flowrates through the system are implemented using the load cell 116, load cell 118, and load cell 402. Load cell 116 measures a weight change over time of the effluent. Load cell 118 measures a weight change over time of the replacement fluid. Load cell measures a weight change of the total weight change over time of the replacement fluid and effluent. Load cells 116, 118, and 402 send a signal to a control system 404. The control system 404 can calculate and convert to the real-time flow rate change for (i) replacement fluid rate, (ii) ultrafiltration (effluent) rate, (iii) net-filtration rate to determine the ultrafiltration or back-filtration over time.

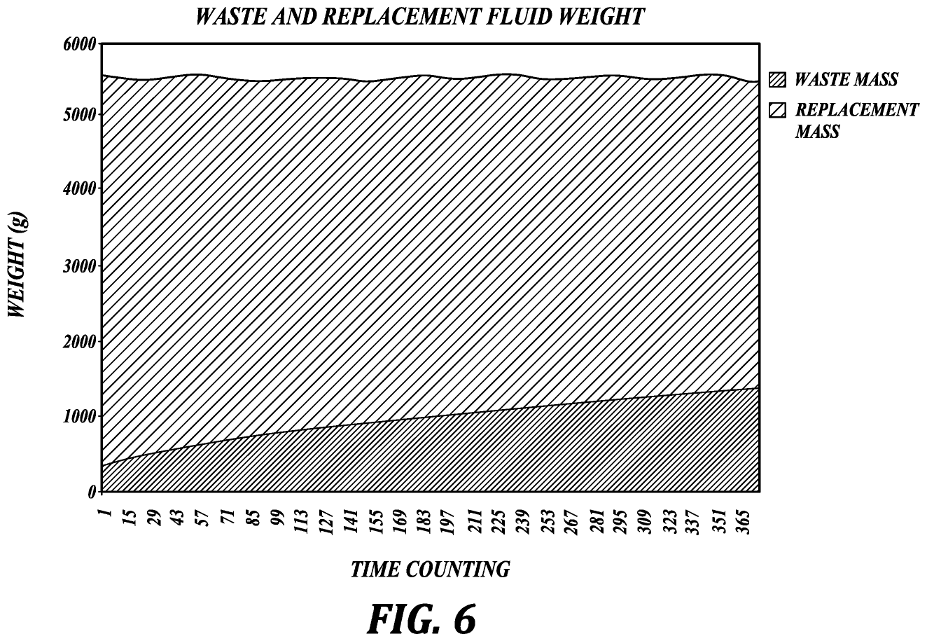

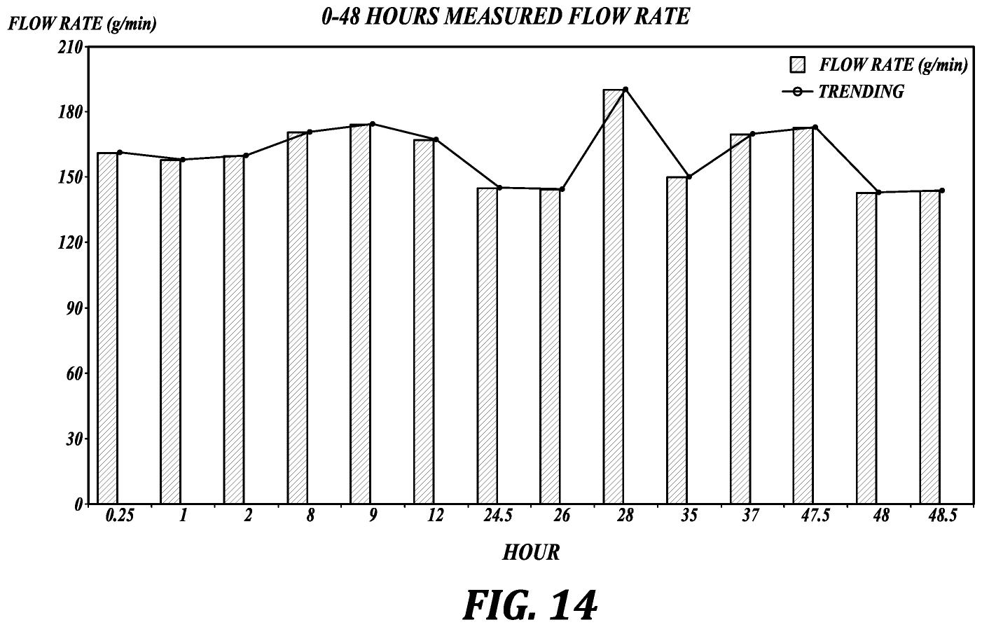

FIG. 6 is a graph showing one example of the testing result of the weight management system over time. The portion of the graph above the line represents the reduction of the replacement fluid while the portion of the graph below the line represents the increasing of the ultrafiltration mass. In the example embodiment, the total weight of replacement fluid and effluent remains constant representing there is no net-filtration over the time. FIG. 14 is a graph showing the stability of the flowrate of the blood using the load cells 116, 118, 402 and control system 404. FIG. 14 shows the flowrate that is controlled generally between 150 and 180 g/min over 48 hours.

FIG. 14 is an example to demonstrate how to control the flow rate with the feedback from the load cell. The blood flow equation is, Q=(mᵢ−mⱼ)/(tᵢ−tⱼ), where Q is the flow rate in unit of mL/min, mᵢ is the mass reading of the load cell at time point i, in the unit of g, mⱼ is the mass reading of the load cell at time point j, in the unit of g, tᵢ and tⱼ is the time point at i and j, respectively, in the unit of seconds.

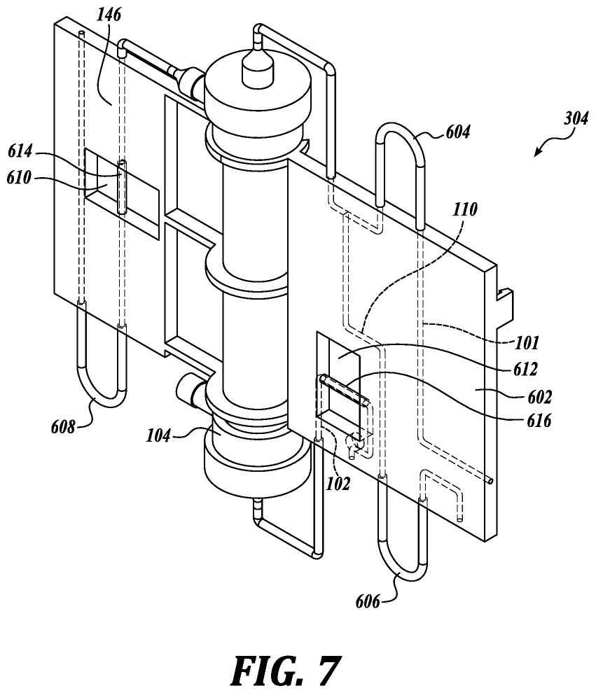

FIG. 7 is a diagrammatical illustration of a disposable cassette layer 304. In one embodiment, the cassette layer 304 can be made replaceable and/or disposable. The cassette layer 304 includes a board 602 that includes the dialyzer 104. The board 602 can be made using polymers, including biodegradable polymers.

In one embodiment, the board 602 is capable of automatic loading tubing for blood leak detectors, air bubble detectors, instruments, peristaltic pumps, and the like, in the system through a user-friendly, one touch tubing loading program.

The board 602 includes tubing channels that are provided within the board 602. The tubing channels formed within the board 602 can be used instead of tubes and piping. The tubing channels may terminate at the edge of the board for making connections to eternal tubes or pipes that connect to other non-disposable system components. The tubing channels within the board 602 include the effluent line 146, the replacement fluid line 110, the blood inlet line 101, the blood outlet line 102. The lines 146, 110, 101, and 102 can terminate at the edge of the board 602 and tubing or piping can be used to complete the lines, as illustrated in FIG. 1. For example, the blood inlet line 101 and blood outlet line 102 from the board 602 can be connected to exterior tubing extending the blood inlet line 101 and the blood outlet line 102 to the patient where the lines can include a catheter for drawing and returning blood to a patient.

The board 602 may include openings 610, 612 to allow instruments, including valves, detectors, and the like, to be placed in the appropriate location of each channel. In each opening 610, 612, tubing 614, 616 or piping can be used to connect the channel across the openings 610, 612. Instruments, valves, and detectors can be non-contact sensors and valves so that the sensors and valves are not in contact with the fluids within the channels. The sensing element may be placed against the tubing 614, 616. For peristaltic pumps, for example, a flexible tubing can be connected to the channel terminations at the edge of the board 602 so that the tubing is used to form loops 604, 606, 608 exterior to the board 602 that allow the placement of a peristaltic pump on each tubing loop.

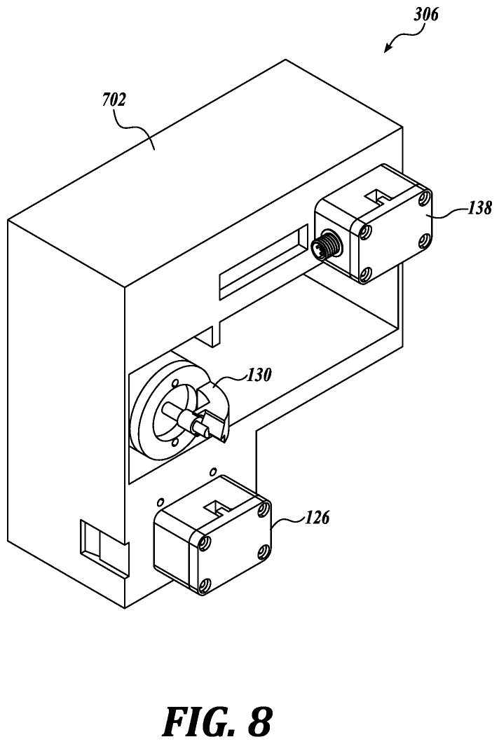

In one embodiment, non-disposable instrument components are installed on the sensor and electrical components layer 306. A section of the sensor and electrical components layer 306 is illustrated in FIG. 8. The sensor and electrical components layer 306 includes a lifter block 702 to which the sensor and electrical components are fitted. In one embodiment, the lifter block 702 supports the various sensors and electrical components on the side that is to be placed juxtaposed to the cassette 602 to locate the sensors and electrical components at the appropriate location in each channel. At all events, the lifter block 702 is used for locating the sensors and the electrical components into the appropriate flow lines to create the functionality illustrated in FIG. 1.

In the illustrated embodiment of the lifter block 702, the blood leak detector 138, the air bubble detector 126, and the pinch valve 130 are shown attached to the lifter block 702. The locations of the sensors and electrical components on the lifter block 702 matches to specific locations on the cassette board 602, so that the board 602 can be joined to the lifter block 702.

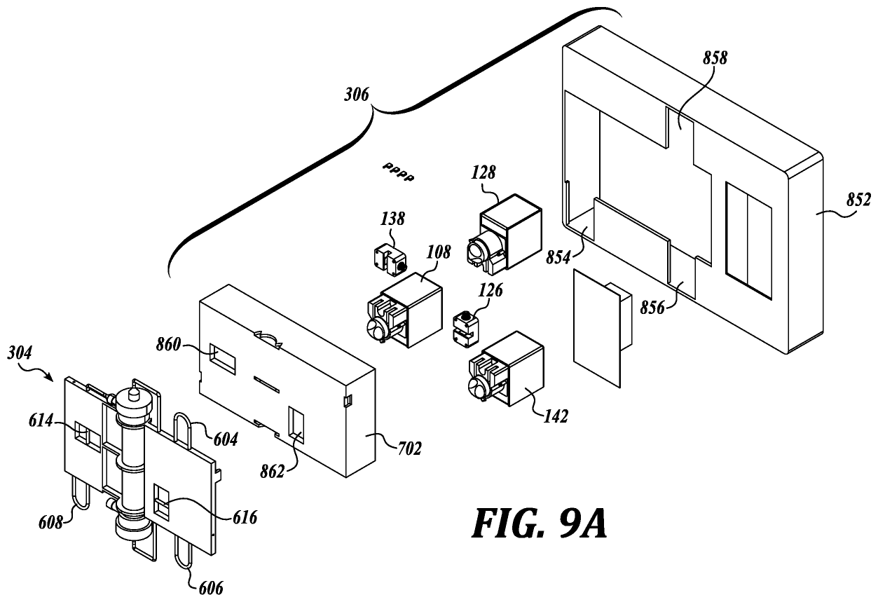

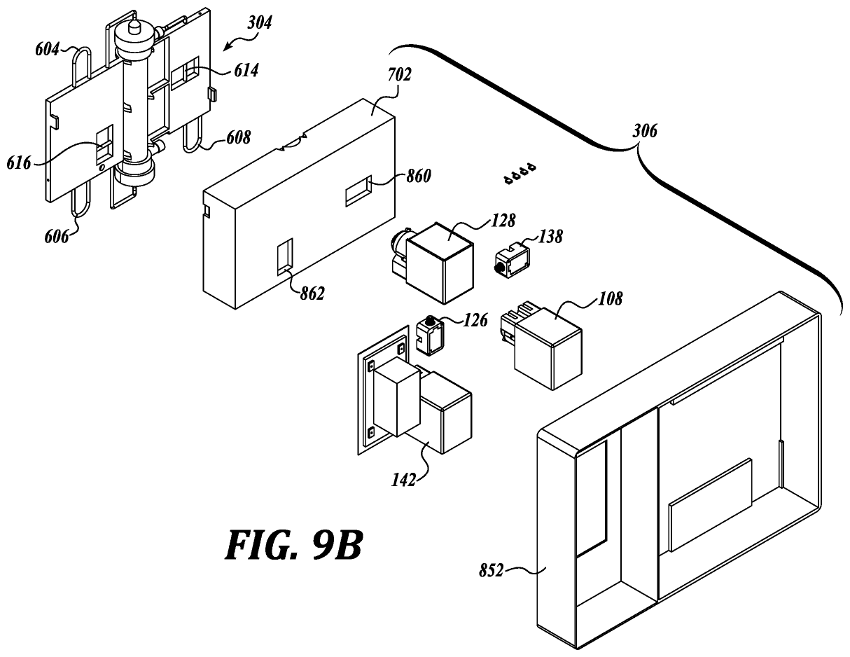

Referring to FIGS. 9A and 9B, a diagrammatical illustration shows an exploded view of the sensor and electrical components layer 306. FIGS. 9A and 9B shows a mounting baseplate 852 that is designed to hold the lifter block 702 to ensure the smooth connection between the disposable cassette layer 304 and the sensor and electrical components layer 306. The mounting baseplate 852 includes compartments 854, 856, 858, respectively for pumps 108, 142, 128, as well as a compartment for the lifter block 702. In turn, the lifter block 702 includes apertures 860, 862 for sensors 138, 126, respectively, to pass through the lifter block 702 and make connections to the disposable cassette layer 304. In particular, sensors 138 and 126 include slots that accept the tubing 614 and 616. The disposable cassette layer 304 includes the tubing loops 604, 606, and 608 that accept the peristaltic pumps 128, 142, and 108.

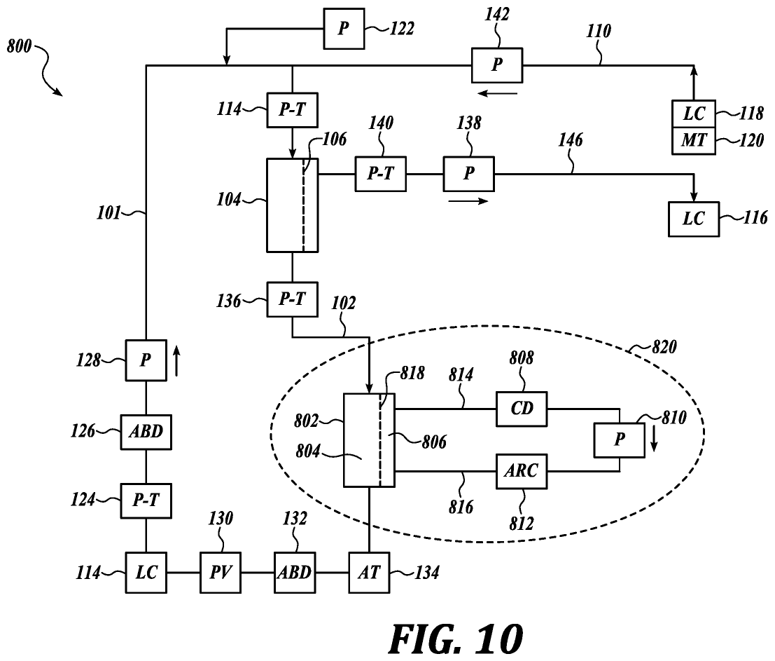

Referring to FIG. 10, an embodiment of a hemopurification system 800 including a liver module 820 is illustrated. In one embodiment, the hemopurification system 800 is similar to the hemopurification system of FIG. 1; however, the hemopurification system 800 includes a second dialyzer to remove protein-bound toxins from the blood. In FIG. 10, like reference numbers represent the same components as described for the system in FIG. 1. In one embodiment, the hemopurification system 800 includes a portable and automated hemopurification system configured to remove one or more toxins from blood of a patient and treat imbalanced body fluid in blood caused by disease of vital organs, such as liver and kidney.

The hemopurification system 800 of FIG. 10 includes a blood inlet line 101 configured to deliver blood from the patient; a blood outlet line 102 configured to return blood to the patient; a first dialyzer 104 configured to remove one or more uremic toxins from blood of a patient, the first dialyzer includes a first dialyzing membrane 106, an inlet end that receives blood provided by the blood inlet line, and an outlet end that provides blood towards the blood outlet line; a negative-pressure pump 138 configured to apply negative pressure across the first dialyzing membrane, to provide ultrafiltration of uremic toxins from the blood; a replacement fluid line 110 configured to deliver replacement fluid to the blood prior to or after the first dialyzer; a second dialyzer 802 configured to remove one or more protein-bound toxins from the blood of the patient, the second dialyzer includes a second dialyzing membrane 818 separating the blood from an solute-based dialysate, wherein the solute-based dialysate draws the toxins across the second dialyzing membrane from the blood to the solute-based dialysate.

In one embodiment, the second dialyzer 802 can include a housing to hold the second dialyzing membrane 818. The second dialyzing membrane 818 separates two compartments 804, 806 within the housing. Blood flows within the compartment 804, and the solute-based dialysate flows within the compartment 806. The dialyzing membrane 106 can be a hollow fiber permeable membrane or a flat permeable membrane or any combination. The second dialyzer 802 includes a first inlet and a first outlet in compartment 804 for the blood, a second inlet and a second outlet for the solute-based dialysate in compartment 806. In the illustrated embodiment, the first inlet of the second dialyzer 802 is connected to the outlet end of the first dialyzer 104.

Referring to FIG. 10, the solute-based dialysate is recirculated in a loop by pump 810. The pump 810 can be a peristaltic pump. The pump 810 can be connected to the automatic control system that controls the flow rate of solute-based dialysate. From pump 810, the solute-based dialysate passes through a solute, such as albumin, container 812 to add the solute, such as albumin, to the dialysate. The solute-based dialysate then passes into the second dialyzer 802 on the side of the dialyzing membrane 818 that is opposite to the blood. In one embodiment, the solute-based dialysate is pure water or any solution of pure water with electrolytes and salts, such as bicarbonate or sodium chloride, with the addition of albumin. In an embodiment, the solute can be albumin. The albumin can be human serum albumin, bovine serum albumin, porcine serum albumin, or a combination.

With the use of solute-based dialysate, the second dialyzer 802 can remove small and medium-sized toxins, such as protein-bound toxins. The proteins can transfer across the second dialyzing membrane 818 to bind to the albumin. After exiting the second dialyzer 818, the solute-based dialysate is passed through a filter 808, such as a charcoal detoxifier 808. The charcoal detoxifier 808 regenerates the solute-based dialysate to be continuously used in the second dialyzer 818.

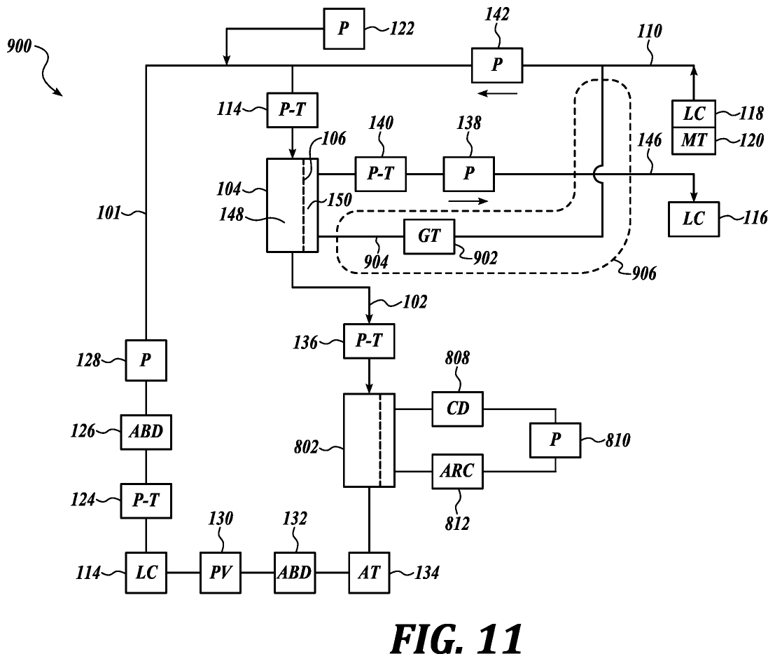

Referring to FIG. 11, an embodiment of a hemopurification system 900 with a lung module 906 is illustrated. The hemopurification system 900 is similar to the hemopurification system of FIG. 10, wherein like reference numbers represent the same components as described for the systems in FIGS. 1 and 10. In one embodiment, the hemopurification system 900 includes a portable and automated hemopurification system configured to remove one or more toxins from blood of a patient and treat imbalanced body fluid in blood caused by disease of vital organs, such as liver, kidney, and lung.

The hemopurification system 900 of FIG. 11 includes a blood inlet line 101 configured to deliver blood from the patient; a blood outlet line 102 configured to return blood to the patient; a first dialyzer 104 configured to remove one or more uremic toxins from blood of a patient, the first dialyzer includes a first dialyzing membrane 106, an inlet end that receives blood provided by the blood inlet line, and an outlet end that provides blood towards the blood outlet line; a negative-pressure pump 138 configured to apply negative pressure across the first dialyzing membrane, to provide ultrafiltration of uremic toxins from the blood; a replacement fluid line 110 configured to deliver replacement fluid to the blood prior to or after the first dialyzer; a second dialyzer 802 configured to remove one or more protein-bound toxins from the blood of the patient, the second dialyzer includes a second dialyzing membrane 818 separating the blood from an solute-based dialysate, wherein the solute-based dialysate draws the toxins across the second dialyzing membrane from the blood to the solute-based dialysate, and wherein the first dialyzer 104 includes a first chamber 148 separated from a second chamber 150 by the first dialyzing membrane 106, wherein a gas line 904 is connected to an inlet of the second chamber to introduce a gas. The gas can include oxygen and carbon dioxide, such that the oxygen can transfer across the first dialyzing membrane 106 to raise the oxygen level in the blood.

In one embodiment, the oxygen module 906 is implemented using the first dialyzer 104 which includes a first inlet connected to the blood inlet line 101 and a first outlet connected to the blood outlet line 102, a second inlet connected to the replacement fluid line 110, and a second outlet connected to the negative pressure pump 108, wherein the gas is introduced into the replacement fluid line 110 before the second inlet of the first dialyzer. As can be seen, unlike the first dialyzer 104 of FIG. 1, the first dialyzer 104 of FIG. 11 has an inlet in addition to the outlet on the dialysate side of the dialyzing membrane 106. The second inlet is used to introduce a portion of the replacement fluid with oxygen. A gas tank 902 can be connected to the replacement fluid line 110 to add oxygen.

In one embodiment, the automatic control system 404 is also used for controlling a method for automatic priming and a rinse-back process. Referring to FIG. 12, a first step of automatic priming is illustrated. The automatic control system 404 operates the pumps 108, 128, and 142 in the direction shown. As can be seen pumps 128 and 142 are operated in the opposite direction as compared to FIG. 1, while pump 108 is operated in the same direction as in FIG. 1. The effluent line 146 is connected to the replacement fluid container via a first bypass line 1202. A three-way control valve 1204 is used to control the direction of the effluent to the replacement fluid container or to the effluent waste container. The blood inlet line 101 is also connected to the replacement fluid container via a second bypass line 1206. A three-way control valve 1208 is used to control the direction of the fluid in the blood inlet line 101. A third bypass line 1210 connects the blood outlet line 102 to the replacement fluid container. A three-way control valve 1212 is used to control the direction of the fluid in the blood outlet line 102.

In the priming step, the flow is a continuous loop in the opposite direction compared to FIG. 1. Flow through the dialyzer 104 is in the opposite direction, whereby fluid enters through the outlet end and flows out of the dialyzer 104 into the blood inlet line 101 and the replacement fluid line 110. Fluid also flows out of the dialyzer 104 through the effluent but bypassed the effluent container and flows into the replacement fluid container. The three lines 110, 146, and 101 flow into the replacement fluid container. Then, from the replacement fluid container, the flow passes into a bypass line to blood outlet line 102 and into the dialyzer 104 outlet end, then back again to the lines 110, 146 and 101.

Referring to FIG. 13, a diagram of the rinse step is illustrated. In the second rinse step, the pumps 108 and 142 are turned off, while the pump 128 is reversed compared to FIG. 12. The three-way control valves 1204, 1208, and 1212 are in the bypass position similar to FIG. 12. The flow is pumped using pump 128 in a continuous loop through the blood inlet line 101 through the dialyzer 104. Then, out of the dialyzer 104 through the blood outlet line 102 to the replacement fluid container through the bypass line 1210. Then, from the replacement fluid container through the bypass line 1206 to the blood inlet line 101 and back to the dialyzer 104.

Through the control of the three-way valves and change of the pumps' flow direction, the priming and rinse-back process is achieved by a single button, one-touch protocol via the automatic control system 404.

In one embodiment, the replacement fluid container includes a medical-fluid warmer 120. The medical-fluid warmer 120 provides an efficient method and device for medical fluid warming that can be integrated into the hemopurification system or other medical devices. This medical-fluid warmer 120 will offer the precise temperature monitoring and rapid warming functionality for any kind of fluid being delivered within medical devices. The functionality of temperature monitoring will be achieved using multiple thermocouples configuration and feedbacks. As for fluid warming, the medical-fluid warmer 120 will only use simple heat conduction and convection from a low wattage heat source within specially designed heating cassette. This device will offer the capability of warming up the fluid from room temperature to a general temperature close to human body (37° C.) at a maximum flow rate of 50 ml/min using only 60 watts. Additionally, the disposable cassette especially for the heating will offer the convenience of disinfection process between each treatment since it does not require any sterilization for the device. With the characteristics of rapid heating using low wattage, compact design, and disposable tubing configuration, the medical fluid heating will be able to be integrated into any of the current medical devices that require precise and rapid fluid heating solutions.

Overall, the medical-fluid warmer has potential applications, such as: 1. Replacement Fluid Warming During Continuous Renal Replacement Therapy (CRRT): to achieve desired warming precision during CRRT treatment using portable device. 2. Emergency Department (ED) IV Fluid Warming: to improve comfort for patient in ED setting and minimize the chance of hypothermia after rapid IV therapy. 3. Blood Warmer: to achieve precise temperature control for any blood delivery as well as convenience and safety of noninvasive warming solution.

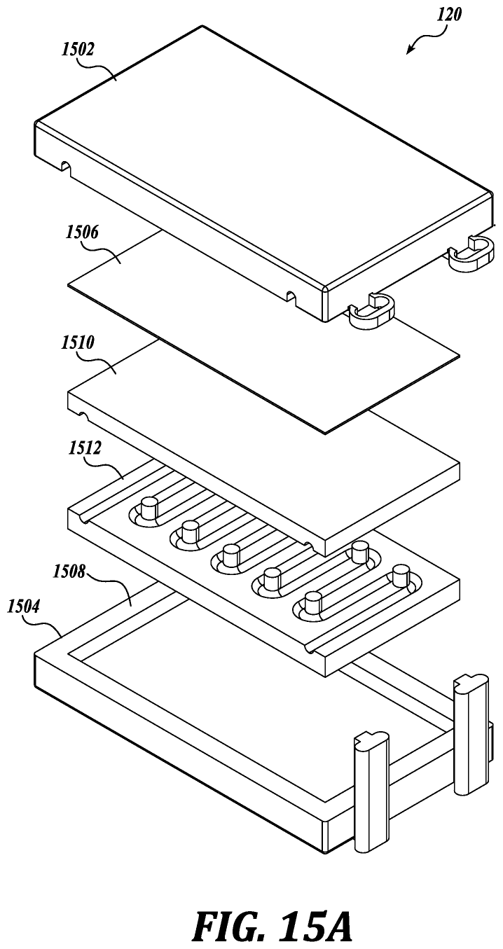

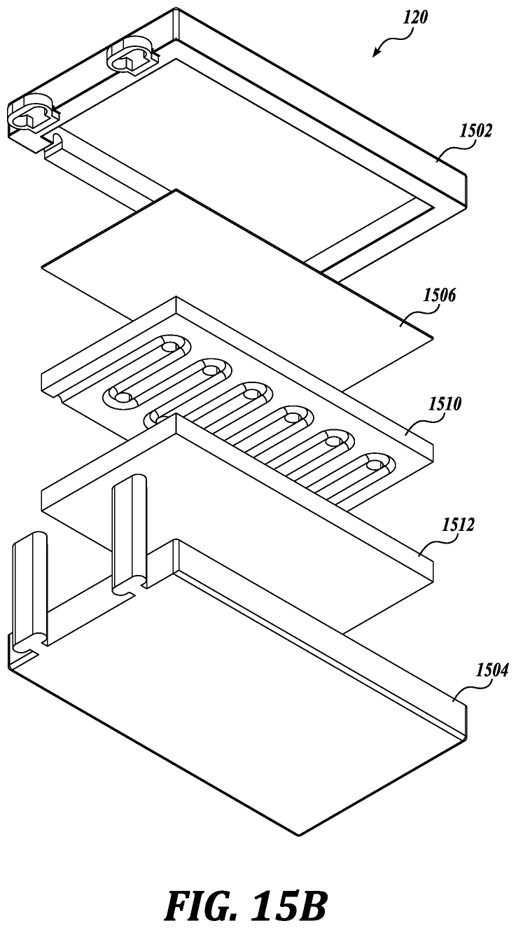

FIGS. 15A and 15B are diagrammatical illustrations of one embodiment of a medical-fluid warmer 120. The medical-fluid warmer 120 includes an insulation top cover 1502, an insulation bottom cover 1504, a top heating pad 1506 juxtaposed beneath the top insulation top cover 1502, a bottom heating pad 1508 juxtaposed above the insulation bottom cover 1504. The medical-fluid warmer 120 includes a top copper conductive panel 1510 and a bottom copper conduction panel 1512. The outside surface, which can be flat, of the top copper conductive panel 1510 faces the top heating pad 1506. The outside surface, which can be flat, of the bottom copper conductive panel 1512 faces the bottom heating pad 1508. The inside surfaces of the both the top and bottom copper conductive panels 1510, 1512 can be machined or otherwise formed to provide a serpentine flow channel that winds back-and-forth across the copper conductive panels 1510, 1512. An inlet and an outlet is provided to for the flow channel that allows fluid into and out of the serpentine channel. The top and bottom copper conductive panels 1510, 1512 are joined in a fluid-tight manner to prevent leaks. The top heating pad 1506 and the insulation top cover 1502 are placed above, and the bottom heating pad 1508 and the insulation bottom cover 1504 are placed below, and the layers are then fastened.

Representative embodiments may include, but, are not limited to the following embodiments.

1. A portable and automated hemopurification system configured to remove one or more uremic toxins from blood of a patient, comprising the following elements:
a primary dialysis circuit comprising
a blood inlet line configured to deliver blood from the patient;
a blood outlet line configured to return blood to the patient; and
a dialyzer with a dialyzing membrane, an inlet end that receives blood provided by the blood inlet line, and an outlet end that provides blood towards the blood outlet line;
a negative-pressure pump configured to apply negative pressure across the dialyzing membrane, to provide ultrafiltration of uremic toxins from the blood;
a replacement fluid line configured to deliver replacement fluid to the blood; and
pumps configured to drive blood and fluidic flow in the system.

2. The portable hemopurification system of Embodiment 1, wherein the negative-pressure pump applies a negative pressure across the dialyzer membrane to draw the uremic toxins across the dialyzing membrane.

3. The portable hemopurification system of Embodiment 1, wherein the dialyzer includes a first compartment and a second compartment separated by the dialyzing membrane, and the first compartment includes the inlet for the blood inlet line and the outlet for the blood outlet line, and the second compartment includes no inlet and an outlet for a line to the negative-pressure pump,

4. The portable hemopurification system of Embodiment 1, wherein the system operates using Continuous Veno-Venous Hemofiltration (CVVH), intermittent veno-venous hemofiltration, continues arteriovenous hemofiltration.

5. The portable hemopurification system of any of the preceding Embodiments, wherein dimensions of suitcase containing the portable hemopurification system are equal to or less than 55 cm×40 cm×20 cm.

6. The portable hemopurification system of any of the preceding Embodiments, wherein a weight of the portable hemopurification system is 25 pounds or less.

7. The portable hemopurification system of any of the preceding Embodiments, further comprising a real-time flowrate and weight management system, comprising:
a replacement fluid load cell configured to measure weight of replacement fluid at the start, end, and throughout the entire treatment;
an effluent load cell configured to measure weight of effluent produced by the negative-pressure pump at the start, end, and throughout the entire treatment;
a total weight load cell configured to measure the total weight of replacement fluid and effluent at the start, end, and throughout the entire treatment; and
a controller configured to use changes in weight from the replacement fluid load cell, the effluent load cell, and the total weight load cell to determine:
a replacement fluid rate;
an effluent rate;
a net-filtration rate; and
combinations thereof.

8. The portable hemopurification system of any of the preceding Embodiments, wherein a control system tracks the replacement fluid rates, effluent rates, and checks any variations from the total weight. An alarm will be triggered if there are any variations.

9. The portable hemopurification system of any of the preceding Embodiments that permits corrective measures to be taken automatically.

10. The portable hemopurification system of any of the preceding Embodiments, further comprising a disposable cassette system that contains the dialyzer, pressure sensor connectors, temperature sensor connectors, anti-coagulant connectors, and at least portions of the blood inlet line and the blood outlet line.

11. The portable hemopurification system of any of the preceding Embodiments, further comprising a non-disposable layer that includes sensors and electrical components configured to monitor and operate the system.

12. The portable hemopurification system of any of the preceding Embodiments, further comprising sensors selected from pump sensors, blood leak detection (BLD), air-bubble detection (ABD), pressure sensors, load cells, temperature sensors, pinch valves, and combinations thereof.

13. The portable hemopurification system of any of the preceding Embodiments, further comprising an automatic priming, rinse-back system.

14. The portable hemopurification system of any of the preceding Embodiments, wherein the replacement-fluid line further comprises a fluid warmer.

15. The portable hemopurification system of any of the preceding Embodiments, wherein the medical-fluid warmer comprises a disposable heating cassette comprising a serpentine fluidic path in thermal communication with at least one heating element.

16. The portable hemopurification system of any of the preceding Embodiments, wherein at least one pump is a peristaltic pump.

17. The portable hemopurification system of any of the preceding Embodiments, wherein at least one pump is a syringe pump.

18. A method of hemopurification of a patient's blood, comprising flowing blood from the patient through a portable hemopurification system of any of the preceding Embodiments, and returning the blood to the patient.

19. A fluid warmer, comprising a disposable heating cassette comprising a serpentine fluidic path in thermal communication with at least one heating element.

20. The medical-fluid warmer of Embodiment 19, further comprising an insulation top cover, an insulation bottom cover, a top heating pad juxtaposed beneath the top insulation top cover, a bottom heating pad juxtaposed above the insulation bottom cover, a top copper conductive panel juxtaposed below the top heating pad, a bottom copper conduction panel juxtaposed above the bottom heating pad, wherein surfaces of the top and bottom copper conductive panels facing one another include the serpentine flow channel.

21. The medical-fluid warmer of Embodiment 19 or 20, wherein the fluid warmer is configured for uses as Replacement Fluid Warming During Continuous Renal Replacement Therapy (CRRT); Emergency Department (ED) IV Fluid Warming; or a blood warmer.

22. A hemopurification system configured to remove one or more toxins from blood of a patient and treat imbalanced body fluid in blood caused by disease of vital organs, such as liver, kidney, and lung, comprising:
a blood inlet line configured to deliver blood from the patient;
a blood outlet line configured to return blood to the patient; and
a first dialyzer configured to remove one or more uremic toxins from blood of a patient, the first dialyzer includes a first dialyzing membrane, an inlet end that receives blood provided by the blood inlet line, and an outlet end that provides blood towards the blood outlet line;
a negative-pressure pump configured to apply negative pressure across the first dialyzing membrane, to provide ultrafiltration of uremic toxins from the blood;
a replacement fluid line configured to deliver replacement fluid to the blood;
a second dialyzer configured to remove one or more protein-bound toxins from the blood of the patient, the second dialyzer includes a second dialyzing membrane separating the blood from an solute-based dialysate, wherein the solute-based dialysate draws the protein bounded toxins across the second dialyzing membrane from the blood to the solute-based dialysate.

23. The hemopurification system of Embodiment 22, wherein the second dialyzer includes a first inlet and a first outlet for the blood, a second inlet and a second outlet for the solute-based dialysate, wherein the first inlet of the second dialyzer is connected to the outlet end of the first dialyzer.

24. The hemopurification system of Embodiment 22 or 23, wherein the solute-based dialysate from the second outlet of the second dialyzer is regenerated and introduced to the second inlet of the second dialyzer.

25. The hemopurification system of anyone of Embodiments 22 to 24, wherein the solute-based dialysate is passed through charcoal before introducing to the second inlet.

26. The hemopurification system of anyone of Embodiments 22 to 25, wherein the solute-based dialysate includes human serum albumin, bovine serum albumin, porcine serum albumin, other solutes, or a combination.

27. The hemopurification system of anyone of Embodiments 22 to 26, wherein an additional pump is connected by tubing to the inlet of the regeneration device or charcoal/resin column. This additional pump connects to a dialysate reservoir. A tubing line connects the outlet to a waste container. Solute-based dialysate may be pumped through a saturated charcoal/resin column or regeneration device to restore its toxin binding capacity.

28. The hemopurification system of anyone of Embodiments 22 to 27, wherein the first dialyzer includes a first chamber separated from a second chamber by the first dialyzing membrane, wherein a gas line is connected to the second chamber to introduce gas.

29. The hemopurification system of anyone of Embodiments 22 to 28, wherein the first dialyzer includes a first inlet connected to the blood inlet line and a first outlet connected to the blood outlet line, a second inlet connected to the replacement fluid line, and a second outlet connected to the negative pressure pump, wherein the gas is introduced into the replacement fluid line before the second inlet.

130. The hemopurification system of anyone of Embodiments 22 to 29, wherein the gas is oxygen.

31. The hemopurification system of anyone of Embodiments 22 to 30 where other physiological gases such as carbon dioxide or nitrogen may be used.

### EXAMPLES

In vitro experiments were conducted to establish the efficacy and safety of an albumin recirculating regenerating system. Based on these experiments, a liver dialysis system was developed that uses recirculating regenerating albumin dialysate that can be used as a liver dialysis system. Additionally, the combined hemodialysis and albumin dialysate system provides both liver and kidney dialysis simultaneously. This Modified Albumin Hemodialysis (MAHD) is further described in this EXAMPLES section.

### Methods

In Vitro Studies: Work was replicated showing albumin dialysate can remove bilirubin in a benchtop model and charcoal columns can regenerate albumin dialysate (10-12). In addition, a negative control condition was tested to determine the capacity of pure dialysate to remove toxins. In the negative control, there was no bovine serum albumin (BSA) on the dialysate side.

Reagents for in vitro study: 97% pure bilirubin was purchased from Alfa Aeser (A17522). 98% pure BSA was purchased from Millipore Sigma (A7906). 98% pure indoxyl sulfate was purchased from Millipore Sigma (I3875). 98% pure cholic acid was purchased from Millipore Sigma (C1129). 99.99% pure manganese chloride was purchased from Millipore Sigma (203734). 99.0% pure copper chloride was purchased from Millipore Sigma (307483). 98% pure creatinine was purchased from Millipore Sigma (C4255). 1 Norm hydrochloric acid was purchased from Fisher Chemical (SA48). 1 Norm sodium hydroxide was purchased from Titristar (SX0607H-6). 99.9% Anhydrous sodium carbonate was purchased from Millipore Sigma (1063920500). 99.9% pure DMSO was purchased from Millipore Sigma (MX1458-6). Dialysate was made by mixing the Centrisol MB-330-L Bicarbonate Concentrate from Minntech with the NaturaLyte 08-3301-2 Acid Concentrate from Fresenius according to the instructions on the packaging. Bilirubin measurement reagent was purchased from Beckman Coulter (OSR6112). Albumin measurement reagent was purchased from Beckman Coulter (OSR6102).

Benchtop Setup: BSA and toxins (listed below) are added to the blood side. BSA is chosen because it has 76% homology to HSA and is frequently used as a lower cost substitute. Differences between the two, human and bovine serum albumin, may be a confounding factor. A blood side BSA concentration of 2 g/dL is a clinically relevant marker of severe liver failure. The blood side flow rate was 180 mL/min. The dialysate side contained 2 g/dl concentration of BSA and the flow rate was 90 mL/min. This was predicted to be the optimal flow rate. The dialyzers used were the Fresenius F6HPS dialyzer (polysulfone, surface area of 1.3 m²) or the Baxter CT110G (cellulose triacetate, surface area of 1.1 m²). For the in vitro work only, dialyzers were cleaned and reused according to established clinical protocols. The pH of the solutions was adjusted to a value between 7.1 and 7.5 using NaOH and HCl, pH was measured by a pHoenix XL meter from MesaLabs. The dialysate side container was a sealed, rigid flask. This was intended to prevent ultrafiltration. Prevention of ultrafiltration was verified by examining the volume before and after the experiment and by pressure measurements. Pressure was measured by Omega PX409-015GUSBH or Honeywell RSCDRRE015PGSE3 sensors. Temperature was measured by K type thermocouples connected to a Measurement Advantage DAQ and maintained near 37° C. on both sides using hotplates.

Toxins: Blood analog solution contained BSA, bilirubin, cholic acid, creatinine, indoxyl sulfate, copper, and manganese. Their binding sites differ from that of bilirubin (17-19). Including multiple toxins accurately represents the complex milieu in liver failure. For this study, bilirubin and BSA were measured, and other toxins were not. Bilirubin was dissolved as previously described using DMSO and sodium carbonate. BSA was measured to verify that it did not cross the membrane. Bilirubin initial concentration was approximately 20 mg/dL (12).

Assays: Bilirubin and albumin were measured by the AU680 Chemistry Analyzer from Beckman Coulter. To validate the assay in dialysate and for BSA, standard curves and interference were tested. Each substance was measured separately. Simultaneous measurement altered the reported values. At experimentally relevant concentrations, none of the other solution constituents interfered with albumin or bilirubin measurements. A blank (deionized water) was run with each assay. If a sample was not distinguishable from the blank its concentration was set to 0. Each sample was run in triplicate.

Charcoal Column Regeneration: BSA dialysate solutions were regenerated using charcoal as described previously, without applying the pre-concentration method. Briefly, 75 g of charcoal was added to 3 column segments. The charcoal was NORIT RO 0.8. The column was assembled as previously described, except that instead of filters built into the top segment, a 150 μm filter (McMaster-Carr 8991T36) was added on the outlet of the top segment. Priming was done for 48 hours against gravity at 120 mL/min with deionized water. Then, before the experiment, priming was done for 30 minutes with dialysate. Priming volume was determined by comparing the dry weight of the column and filter to the full weight of the assembly. For the trial, the flow direction was reversed {what does this mean?). Samples were taken at the inlet and outlet of the charcoal column. Pressure was also measured at these points. The trial was stopped if pressure difference across the column exceeded 30 mmHg.

Statistics: Comparisons are done using the unpaired two-tailed students t-test without assuming equal variance. A p<0.05 is considered statistically significant for the in vitro study. For charcoal column trials, percent remaining is calculated by dividing the value at time t by the value at time 0 and multiplying by 100%.

Patients:

The patients were all listed for liver transplantation but had been made inactive on the transplant wait list because of their unstable clinical condition.

All patients had multi-organ (>4 system) involvement:
1. Kidney failure with large extracellular volume (ECV) excess and requiring hemodialysis treatment
2. Central nervous system involvement, in deep coma
3. Respiratory failure on ventilatory support
4. Cardiovascular involvement with hypotension requiring vasopressor support
5. Hematological system involvement with active bleeding from multiple sites
6. Calculated MELD in all patients was ≥40

All patients had renal failure with significant volume overload requiring ultrafiltration and HD. Due to hemodynamic instability, these patients had poor tolerance to ultrafiltration using traditional hemodialysis, which made fluid removal and volume control very difficult. There were more women and the average age was 54.2+4.4 (mean±SD).

Liver Dialysis System (MAHD) Description:

A liver dialysis segment (Segment A) was inserted in the arterial line of the Fresenius 2008K hemodialysis machine. The Segment A included various components, each of these are termed as “Liver” (L) in order to separate these from usual hemodialysis machine components. These components included a thin membrane cellulosic dialyzer, Exeltra 210 (Baxter) (termed as Liver Dialyzer, LD); the dialysate ports of the LD were connected to a plastic 10 Liter container (LC) through a tube (LT). The LT was fed through a positive displacement pump (LP) that controlled the flow of dialysate with albumin (albumin dialysate, AD) contained in the LC. The AD was constituted by mixing human serum albumin with citrate dialysate to give a final concentration of K 2.0 mEq/L, Ca 3.0 mEq/L, Na 137 mEq/L, Citrate 2.4 mEq/L, HCO3 37 mEq/L and albumin 2-4 grams/dl. A charcoal detoxifier, Adsorba 300 (Gambro) was attached to the LT and AD was circulated through the detoxifier. The albumin dialysate was recirculated at a rate of 20 ml/min in this closed system.

Blood from the patient first passed through the LD (Segment A) before entering the Segment B or Fresenius 2008K hemodialysis machine. The hemodialyzer used in this segment was Diacap LOPS15 (BBraun). The blood flow rate was between 200-300 ml/min and dialysate flow rate 500 ml/min. The regular hemodialysis with ultrafiltration was conducted in the hemodialysis segment B. The ultrafiltration rate and the dialysate composition of segment B was decided on the basis of clinical condition using standard hemodialysis protocol.

Regeneration of Charcoal Column: The charcoal column was flushed with 500 to 1000 ml of 5% dextrose normal saline solution (D5NS) every 60 minutes. Every hour the albumin pump (LP) was stopped and the D5NS line was unclamped, the three way stop cock was turned to close the albumin dialysate circuit and open the saline rinse circuit so that the saline can enter the charcoal column. Similarly at the outflow end of the charcoal column the stop cock turned to the albumin dialysate circuit and open the connection to the drain bag. After the desired volume of D5NS rinsed the column the rinse lines were clamped, stop cocks were reversed to connect with albumin dialysate, start the albumin dialysate pump and the liver dialysis resumed. The volume of D5NS rinse is based on the change in the intensity of yellow rinse coming out of the charcoal column. The rinsing process usually took about 10 minutes. While the charcoal column is rinsed the hemodialysis continues.

### Results

### In Vitro Results

The percentage of BSA and bilirubin removed after 3 hours of albumin dialysis and negative control in vitro trials was tracked. The BSA dialysis condition used 2 g/dL albumin solution to remove toxins from a 2 g/dL BSA blood analog solution. In the negative control, blood analog solution was dialyzed against dialysate alone without BSA. Ultrafiltration was prevented in both experiments. Measurements were done on the blood and dialysate side. Bilirubin removal was significantly greater in albumin dialysis than in the control (p=0.00055). Albumin was not removed in either condition. Albumin loss did not differ between the two (p=0.31).

The bilirubin and albumin remaining in the BSA dialysate solution flowing through the charcoal column at 120 mL/min was tracked. Over 3 hours, 66% of bilirubin and 87.4% of BSA remained in the solution, the rest was adsorbed by the charcoal. The sample size is not sufficient for statistical inference on this in vitro data.

A charcoal column rinsed with dialysate for 30 minutes performs similarly to a fresh one in terms of bilirubin removal. The Percentage of bilirubin remaining in the solution is calculated from the concentration measured at the inlet and outlet of the charcoal column. The amounts remaining with the use of regenerated columns were approximately equal to the ones obtained with the fresh columns.

### Clinical Outcome

All patients responded to the MAHD therapy with improvement in hepatic encephalopathy, respiratory function, and hemodynamic parameters, such that ventilatory and vasopressor supports were no longer needed. Patients also lost significant volume of extracellular fluid excess. Based on transplantation, patients fell into two groups: in group 1, there were five patients who either eventually underwent liver transplantation (n=4) or recovered liver function (n=1); in group 2, five patients died waiting for a liver transplantation (on the wait list). There were no significant differences between the two groups in terms of age, gender or severity of the disease. The first group received slightly fewer MAHD treatments than the second group (9.6 vs. 14.2, p=0.12). The patients who did not survive were on the liver transplant wait list for an average of 35 days after the initiation of MAHD, with one patient waiting for 73 days before dying from sepsis and bleeding.

Fluid Removal by the Dialytic Procedure: The ability to remove fluid with ultrafiltration during the MAHD procedure was quite remarkable. All patients prior to using MAHD had difficulty in fluid removal with usual dialytic techniques that included intermittent HD, Slow Continuous Ultrafiltration or Slow Low Efficiency Dialysis. During these treatments the weight loss was either minimal (<0.4 Kg) or sometimes there was even weight gain by the intravenous fluid given to treat intra-dialytic hypotension. In the same patients with MAHD treatment ultrafiltration was well tolerated even at a rate of one liter per hour (upper limit set by the protocol).

Dialysate albumin and bilirubin removal: The relationship between dialysate albumin concentration and bilirubin removal was studied in one patient. Varying dialysate albumin concentration was used during nine procedures. The concentration ranged from 2.1 to 3.92 grams/dl. The bilirubin concentration was measured at the inlet of the liver dialyzer and outlet of the dialyzer simultaneously. The amount of bilirubin that moved out of the blood across the dialyzer was calculated by subtracting the outlet concentration from the inlet concentration. There was a strong positive correlation (r=0.78) between the albumin concentration and bilirubin movement across the dialyzer membrane.

Bilirubin Removal with Time of Treatment: Bilirubin concentration in the albumin dialysate was measured at the inlet and outlet of dialysate port of the liver dialyzer at start, 30 minutes, 4, 6 and 10 hours of treatment. As expected, dialysate bilirubin at the inlet increased from zero at the start of treatment and continued to increase throughout the procedure. However, the post dialyzer bilirubin concentration remained higher with a positive delta value (post-dialyzer minus pre-dialyzer concentration). The difference between the inlet and outlet concentration decreased with time representing the effect of saturation of binding sites and decrease in gradient.

### Discussion

Three key observations were established: albumin dialysate removes protein-bound toxins across commonly used dialyzer membranes without a need for a specially designed membrane, charcoal columns can regenerate albumin dialysate by removing clinically relevant toxin(s) (bilirubin), and charcoal column saturation can be prevented by periodic rinsing. Studies had established that for three different bilirubin concentrations, closed loop mode albumin dialysis removes bilirubin across an unmodified low flux Gambro 6LR membrane. Equilibrium is attained in less than 3 hours in all cases, with a half-life of less than 1 hour. Studies had established that activated carbon absorbs albumin-bound bilirubin, cholic acid, and tryptophan with low albumin loss. This maintains the chemical gradient for toxin removal into albumin dialysate. Studies had demonstrated that activated carbon can be regenerated by simple dialysate flow through the column. After a 30-minute regeneration with pure dialysate, a charcoal column adsorbed bilirubin similarly to a fresh one.

Many AOCLF patients develop multi-organ failure, mortality with 3 or more system involvement even with the use of state-of-the-art supportive therapies, is quite high, with a 28-day survival of less than 25% (2, 4). With the deterioration in clinical condition, patients become too unstable for liver transplantation. Many dialytic techniques such as charcoal hemodetoxification, intermittent renal replacement therapy (IRRT) and continuous renal replacement therapy (CRRT) and plasma exchange have been found to be generally ineffective in increasing the probability of surviving to liver transplantation. Commercially available devices such as Molecular Adsorbent Recirculating System (MARS®) and Prometheus® have been shown to be associated with clinical and biochemical improvements but have not shown consistent success as a bridge to liver transplantation in patients with AOCLF and multi-organ failure.

The Modified Albumin Hemodialysis System described, was used in a small group of critically ill unstable patients with AOCLF with multiple organ failure. In these patients the system was found to be safe without any serious adverse events. The ten patients with AOCLF and with severe encephalopathy, requiring ventilator, vasopressor, and dialysis support, who were treated with MAHD, all had significant improvement, leading to extubation, discontinuation of vasopressors, and achievement of significant fluid losses. The 28 day survival of 90% in the 10 patients that used MAHD is better than the 25% survival in published reports on comparable patients

Patients with liver and renal failure suffer the consequence of extracellular fluid (ECF) excess leading to the worsening in pulmonary, cardiac and general condition. Usual diffusion dialysis is ineffective in removing the fluid excess because of hemodynamic instability associated with attempted ultrafiltration. One of the effects of the use of MAHD, as observed in the first patient and consistently thereafter, was the tolerance to ultrafiltration without hemodynamic instability. The same patients who had failed ultrafiltration attempts while using traditional hemodialysis or continuous renal replacement therapy were able to tolerate ultrafiltration rate as high as 12 ml/kg/hour (the maximum UF rate permitted by protocol). The reason for this difference between tradition HD and MAHD is unclear, but it may have been caused by the removal of some unknown vaso-depressant substance by the Liver Dialysis segment preventing the significant drops in blood pressure. The improvement associated with plasma exchange in similar patients may be because of clearance of vaso-depressant molecules such as DAMPs, soluble B7 (CD80, 86) and/or angiopectins. The ability to remove large volumes of fluid excess also improves the chances of being eligible for liver transplantation.

The system was used for over 120 procedures, and there were no serious adverse events related to the treatment that required interruption or discontinuation of treatment. When MAHD was started, active bleeding in all patients improved significantly; however, five patients developed bleeding and/or disseminated infections between 12 to 74 days after completion of MAHD therapy, while on the liver transplant wait list.

The artificial liver support system can successfully remove large volumes of excess extracellular fluid. A recent randomized controlled trial (RCT) treated liver failure patients with two hemodiafiltration modalities without sorbent dialysis (30). Their maximum fluid removal rate was 150 mL/h. From their reported treatment durations and fluid removed per hour, it appears they removed on average 3.9 L with CVVHDF and 3.6 L with CAVHDF. The system was able to achieve excess fluid removal of as much as 10 liters per treatment (average 2.6 L/treatment).

Albumin dialysis can raise mean arterial pressure, a precursor for successful fluid removal. A reduction in vasodilatory nitric oxide has been observed in MARS treatments. Nitric oxide itself acts at the point of release and has a half-life of a few seconds. Thus, it is unlikely to be directly removed by albumin dialysis. However, its precursors such as nitrites are removed by MARS. Despite this, none of these studies were able to remove excess fluid with MARS. Most do not report volume changes. A seminal RCT with the earlier DT-BioLogic system reported 1-2 liters of fluid removed.

By combining albumin dialysis and hemodialysis with ultrafiltration, the system is able to achieve extracellular fluid removals an order of magnitude greater than those seen with past systems.

Based on clinical experience of the decrease in efficiency of charcoal column after one hour of use and from the results of in vitro experiments a method was designed to periodically rinse the charcoal column. The hourly rinse with dialysate yielded dark yellow washout fluid that became lighter with continued rinsing. This design worked very well without any complications.

MAHD has many advantages over MARS®:

The simple add-on device uses off the shelf pumps and tubes, with lower initial and disposable costs. The MARS machine costs $24,000 more than the cost of the off the shelf components of MAHD. The disposables cost per treatment is about $3000 for MARS and about $700 for MAHD.

The treatment is similar to usual hemodialysis, so the training of dialysis staff is simpler. The use of the standard HD machine also reduces the cost associated with training nursing and technical staff.

The MAHD system combines albumin dialysis with traditional dialysis in one treatment, thus reducing cost of additional dialysis and fluid removal treatments.

In this study, the 28 day survival of 90% and successful bridge to transplantation in half of the patients is very promising and should be extended to a larger number of patients. The preliminary results suggest that the MAHD system should be studied in a larger group of patients with advanced AOCLF, as a bridge to liver transplantation.

While illustrative embodiments have been illustrated and described, it will be appreciated that various changes can be made therein without departing from the spirit and scope of the invention.

The embodiments of the invention in which an exclusive property or privilege is claimed are defined as follows:

1. A portable and automated hemopurification system configured to remove one or more uremic toxins from blood of a patient, comprising the following elements:
a primary dialysis circuit comprising
a blood inlet line configured to deliver blood from the patient;
a blood outlet line configured to return blood to the patient; and
a dialyzer with a dialyzing membrane, an inlet end that receives blood provided by the blood inlet line, and an outlet end that provides blood towards the blood outlet line;
a negative-pressure pump configured to apply negative pressure across the dialyzing membrane, to provide ultrafiltration of uremic toxins from the blood;
a replacement fluid line configured to deliver replacement fluid to the blood; and
pumps configured to drive blood and fluidic flow in the system.

2. The portable hemopurification system of claim 1, wherein the negative-pressure pump applies a negative pressure across the dialyzer membrane to draw the uremic toxins across the dialyzing membrane.

3. The portable hemopurification system of claim 1, wherein the dialyzer includes a first compartment and a second compartment separated by the dialyzing membrane, and the first compartment includes the inlet for the blood inlet line and the outlet for the blood outlet line, and the second compartment includes no inlet and an outlet for a line to the negative-pressure pump,

4. The portable hemopurification system of claim 1, wherein the system operates using Continuous Veno-Venous Hemofiltration (CVVH), intermittent veno-venous hemofiltration, continues arteriovenous hemofiltration.

5. The portable hemopurification system of any of the preceding claims, wherein dimensions of suitcase containing the portable hemopurification system are equal to or less than 55 cm×40 cm×20 cm.

6. The portable hemopurification system of any of the preceding claims, wherein a weight of the portable hemopurification system is 25 pounds or less.

7. The portable hemopurification system of any of the preceding claims, further comprising a real-time flowrate and weight management system, comprising:
a replacement fluid load cell configured to measure weight of replacement fluid at the start, end, and throughout the entire treatment;
an effluent load cell configured to measure weight of effluent produced by the negative-pressure pump at the start, end, and throughout the entire treatment;
a total weight load cell configured to measure the total weight of replacement fluid and effluent at the start, end, and throughout the entire treatment; and
a controller configured to use changes in weight from the replacement fluid load cell, the effluent load cell, and the total weight load cell to determine:
a replacement fluid rate;
an effluent rate;
a net-filtration rate; and
combinations thereof.

8. The portable hemopurification system of any of the preceding claims, wherein a control system tracks the replacement fluid rates, effluent rates, and checks any variations from the total weight and triggers an alarm if there are any variations.

9. The portable hemopurification system of any of the preceding claims that permits corrective measures to be taken automatically by the control system.

10. The portable hemopurification system of any of the preceding claims, further comprising a disposable cassette system that contains the dialyzer, pressure sensor connectors, temperature sensor connectors, anti-coagulant connectors, and at least portions of the blood inlet line and the blood outlet line.

11. The portable hemopurification system of any of the preceding claims, further comprising a non-disposable layer that includes sensors and electrical components configured to monitor and operate the system.

12. The portable hemopurification system of any of the preceding claims, further comprising sensors selected from pump sensors, blood leak detection (BLD), air-bubble detection (ABD), pressure sensors, load cells, temperature sensors, pinch valves, and combinations thereof.

13. The portable hemopurification system of any of the preceding claims, further comprising an automatic priming, rinse-back system.

14. The portable hemopurification system of any of the preceding claims, wherein the replacement-fluid line further comprises a fluid warmer.

15. The portable hemopurification system of any of the preceding claims, wherein the medical-fluid warmer comprises a disposable heating cassette comprising a serpentine fluidic path in thermal communication with at least one heating element.

16. A fluid warmer, comprising a disposable heating cassette comprising a serpentine fluidic path in thermal communication with at least one heating element.

17. The fluid warmer of claim 16, further comprising an insulation top cover, an insulation bottom cover, a top heating pad juxtaposed beneath the top insulation top cover, a bottom heating pad juxtaposed above the insulation bottom cover, a top copper conductive panel juxtaposed below the top heating pad, a bottom copper conduction panel juxtaposed above the bottom heating pad, wherein surfaces of the top and bottom copper conductive panels facing one another include the serpentine flow channel.

18. The fluid warmer of claim 16 or 17, wherein the fluid warmer is configured for uses as Replacement Fluid Warming During Continuous Renal Replacement Therapy (CRRT); Emergency Department (ED) IV Fluid Warming; or a blood warmer.
